SEQUENCE LISTING (2 sequences)

SEQ ID NO: 1 - protein, 27 aa, Artificial Sequence
  Description of Artificial Sequence Synthetic peptide
STAPPAHGVTSAPDTRPAPGSTAPPKG

SEQ ID NO: 2 - protein, 43 aa, Artificial Sequence
  Description of Artificial Sequence Synthetic polypeptide
TSAPDTRPAPGSTAPPAHGVTSAPDTRPAPGSTAPPAHGVSSL

(19) United States
(12) Patent Application Publication
(10) Pub. No.: US 2012/0034294 A1
(43) Pub. Date: Feb. 9, 2012

(54) METHOD OF MAKING SMALL LIPOSOMES

(75) Inventors: Robert A. Dupuit, Edmonton (CA); William J. Reilley, Cranbury, NJ (US)

(73) Assignee: ONCOTHYREON, INC., Seattle, WA (US)

(21) Appl. No.: 13/140,786

(22) PCT Filed: Dec. 17, 2009
(86) PCT No.: PCT/US2009/068499

§ 371 Date: Sep. 6, 2011

Related U.S. Application Data
(60) Provisional application No. 61/138,353, filed on Dec. 17, 2008.

(51) Int. Cl. A61K 9/127; C07F 9/10; A61P 37/04; A61K 38/17; A61K 39/00; B82Y 5/00

(52) U.S. Cl. 424/450; 514/1.1; 514/20.9; 514/21.3; 424/184.1; 554/79; 977/797; 977/907

(57) ABSTRACT

Liposomes of constrained particle size are prepared by substantially continuously mixing substantially continuously flowing streams of water, and of an organic solvent contain lipid(s) capable of forming liposomes, and cooling the mixture so liposomes form, the ratio of the flow rate of the stream of water to the flow rate of the stream of organic solvent, and the rate of cooling of said mixture, being controlled so as to obtain a preparation of liposomes such that at least about 90% of the liposomes are of a particle size less than about 200 nm.

This application claims the benefit under 35 USC 119(e) and the Paris Convention of U.S. provisional application 61/138,353, filed Dec. 17, 2008, which is hereby incorporated by reference in its entirety.

### FIELD OF THE INVENTION

The present invention relates generally to the field of liposomal vaccine production.

### SUMMARY OF THE INVENTION

It is the goal of the invention to provide liposomes that are less than about 200 nm in size.

It was surprisingly found that, employing a method of liposome formation that includes mixing an organic liquid (wherein lipids are dissolved) and water, the concentration of organic solvent as well as rapid cooling of the resulting mixture are crucial for the formation and maintenance of consistent liposome size. The present method and apparatus facilitate the commercial and scalable synthesis of homogenous formulations of liposomally-incorporated drug vaccines by mixing a lipid solution, containing lipids dissolved in a water-miscible organic solvent, into flowing water under novel conditions to promote the continuous production of vaccine-quality liposomes. The method employs a continuous mixing system whereby the ratio of flow rates, i.e. ratio of lipid solution flow rate to water flow rate, is kept constant, thereby maintaining a constant percentage of organic solvent in the system. The method further employs a rapid and scale-independent cooling step, that follows formation of liposomes and that prevents an increase in average liposome size. The method further provides an arrangement of pipes that promotes the formation of liposomes of desired size.

In order to produce liposomes that are less than about 200 nm in size, according to the present method the concentration of organic solvent in the organic solvent/water mixture is kept between 5% and 30%, more preferred, between 10% and 25%, most preferred between 10% and 25%; the ratio of flow rates (water/organic solvent) is kept between 19:1 and 3⅓:1, more preferably between 9:1 and 5:1 or between 9:1 and 4-1; and cooling of the liposome mixture is completed (about 55° C. to about 30° C.) in less than 5 hours, more preferred less than 2 hours, most preferred less than 30 minutes, most preferably essentially instantly.

The invention circumvents obstacles in the field, namely batch-to-batch inconsistency, undesired increase in liposome size during cooling, and the requirement for elaborate methods such as ultrasonication or pressurized systems. Liposomes produced according to the invention are suitable for the production of vaccines for human or veterinary use.

### BRIEF DESCRIPTION OF THE DRAWINGS

FIG. 1 is a schematic of the apparatus arrangement with insets depicting the arrangement of the “T”-junction and, optionally, whether a pipe comprises any internal protrusions or baffles to enhance turbulence and thereby facilitate mixing.

FIG. 2 is a flow-chart depicting various parameters of the overall clinical manufacturing process.

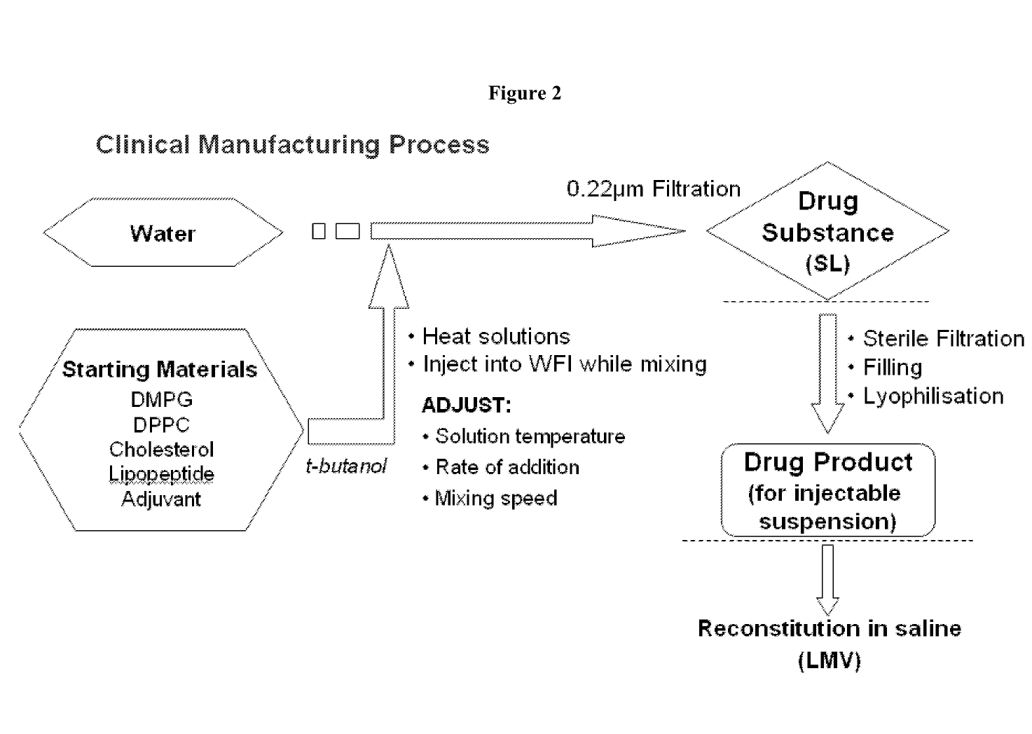

FIG. 3 is a photograph showing the convergence of dye (to mimic lipid/solvent) and water using different diameters of pipes: (A) 9 mm diameters for both pipes; (B) 5 mm (water) and 3 mm (lipid/solvent) pipes.

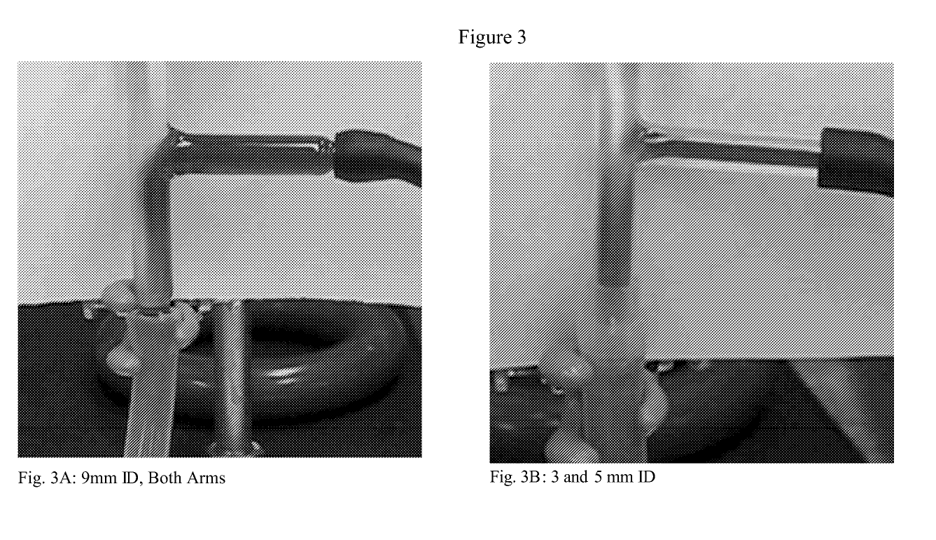

FIG. 4 is a transmission electron microscopy photograph (18K magnification), showing the formation of liposomes carrying MUC-1 peptides using 20% t-butanol produced according to the present method.

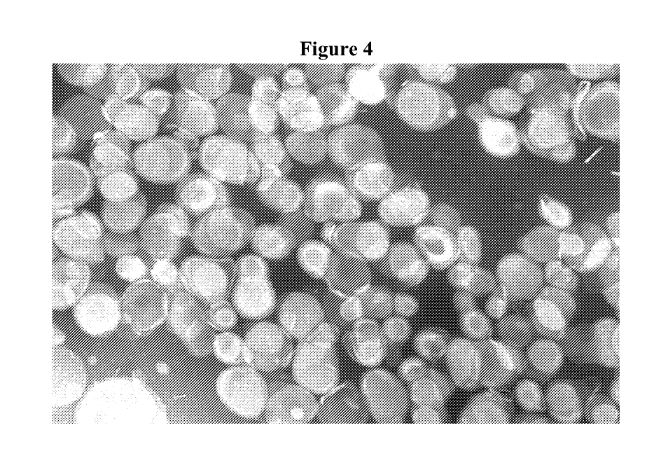

### DETAILED DESCRIPTION OF THE PREFERRED EMBODIMENTS

The present method is adaptable to large-scale, commercial production of formulations of nanoscale liposomes particularly of those that comprise substantially homogenous liposome particle sizes that are no bigger than about 200 nm in diameter. Preferred, more than 90% (volume weighted as determined by dynamic light scattering) of liposomes are less than about 200 nm, most preferred, more than 99% less than about 200 nm. Such sized particles can be readily filter sterilized according to industry-approved clinical manufacturing standards.

A preparation of such homogenously-sized liposomes can be made according to the present invention by controlling the concentration of organic solvent, keeping it essentially constant at, and following, the formation of liposomes. By controlling solvent concentration it is possible to control the size of liposome particles that are formed when the lipid solution and water (or other aqueous solvent suitable for use in liposome formation) converge and interblend.

In this regard, the convergence of lipid solution and water takes place in “midstream” just below the junction of a pipe tubing arrangement through which the solution and water are initially pumped. The lipid solution flows continuously through one pipe and into a continuously flowing stream of water. The two streams can meet at any angle, thus the pipes through which water and lipid solution, respectively, flow might meet at about 90 degrees, or less than 90 degrees. A cloudy mixture of lipid solution and water, the “solvent cloud,” forms just below the junction of the pipes and demarcates the site at which liposomes are believed to be formed.

Furthermore, the degree to which the mixing of the lipid/solvent and water liquids is turbulent can also facilitate liposome formation. Accordingly, a feature of the apparatus and the junction that can be included, but which is not necessary for formation of liposomes, is the incorporation of baffles, internal protrusions, or indentations within the hollow of any of the pipes, which can help to increase turbulence and thereby promote the creation of liposomes. Thus, the creation of high-shear environment at the location where the liquids converge is useful for producing liposomes according to the present invention.

An in-line cooling device that allows for cooling of the mixture during the time between formation of liposomes and entry of mixture into a storage vessel allows for rapid cooling of the liposome mixture. This can be achieved by means of, for example, a cooling jacket, cooling coils, or an ice bath immersing the pipe or other connector through which the liposome mixture flows. Rapid cooling maintains liposome size while during conditions of slow cooling liposome size increases with time at the desired concentration of organic solvent.

By controlling (1) the ratio of water to organic solvent flow rates and (2) the concentration of organic solvent in the mixture and (3) cooling the mixture immediately following formation of liposomes—and optionally (4) using turbulence-enhancing structures, it is possible to continuously produce liposomes that consistently fall within a particular size range.

This arrangement and design therefore avoids the closed and inefficient systems of the prior art that admix together large pre-set volumes of water and lipid/solvent, i.e., from one vat to another (e.g. U.S. patent application Ser. No. 11/185,448). Instead, the present apparatus is a continuously flowing, open system that permits an unending and repeatable process for producing homogenous preparations of liposomes that contain whatever therapeutic substances are incorporated into the lipid solution.

This arrangement is also additionally distinct from prior art apparatuses in that it does not force a pressurized lipid/solvent solution through a discrete orifice or micron sized hole into a stream of water in the form of a pressurized lipid/solvent spray (e.g. U.S. Pat. No. 6,843,942, Wagner et al, 2002, Journal of Liposome Research, 12(3), p. 259-270, U.S. Pat. No. 6,855,277). The present apparatus does not require a “cross-flow injection module” for instance in which the denoted micron sized orifice is made but which otherwise prevents the bulk of the water and lipid liquids from commixing between pipes. That is, the present invention does not forcibly inject a lipid/solvent into water through a tiny hole in co-joining walls of liquid-bearing pipes that otherwise separate the two liquids. To the contrary, the present inventive apparatus and method truly entails the cross-flow of one stream of liquid (water) with another free-flowing stream of liquid (lipid solution) without any such obstruction or pressurized spray. The present invention also does not require any homogenization or sonication as described earlier (e.g. U.S. Pat. No. 6,855,277) for production of liposomes within a defined and consistent size range.

Adding the desired therapeutic compound such as a drug, peptide, or lipopeptide into the lipid solution of the present invention, as well as any other desirable ingredients such as an adjuvant or excipient, facilitates the incorporation of those substances in the liposomes that are formed when the lipid solution converges with the flowing water.

In addition to controlling the concentration of solvent and the ratio of water to lipid solution flow rates, it can also be desirable to heat one or both of the lipid solution and water prior to initiating the flow of each liquid through the denoted piping system. Accordingly, the respective temperatures of the liquids of the present invention can be important criteria for ensuring a consistent and repeatable yield of homogenously-sized, filterable liposomes. Preferred temperature is dependent on the transition temperature for the lipid(s) employed.

The present inventive method allows for operation at a range of practical flow rates. It is a surprising finding that as long as the ratio of flow rates (i.e. ratio of lipid solution flow rate to water flow rate) is kept constant, the speed at which liquids are driven into each other is—within practical ranges—not important. Consequently, the process can be adapted to very small as well as very large total volumes of solution.

Accordingly, factors of the present invention that aid the continuous formation of drug-incorporated, filterable liposomes, include, but is not limited to (1) solvent and solvent concentration; (2) Lipids; (3) ratio of flow rates between lipid solution and water; (4) temperature of the liquids before and at mixing; (5) cooling after the liquids mix and liposomes are formed; 6) the continuous, unobstructed flow of each liquid into each other; and (7) turbulence-inducing means. The following passages elaborate on each of these considerations.

### (1) Solvent and Solvent Concentration

One particular type of solvent of the present invention is a water-miscible organic solvent, such as, but not limited to, lower alkanols, such as methanol, ethanol, propanol, butanol, isoamyl alcohol, isopropanol, 2-methoxy ethanol, and acetone. A preferred solvent of the present invention is butanol or tert-butanol (t-butanol). An organic solvent is useful for dissolving lipids and drug or bioactive agents which then, according to the present invention, is streamed into flowing water, or an aqueous medium, to form the liposomes disclosed herein which incorporate the drug or agent.

One consideration for producing liposomes that fall within a particular size range is the concentration of water miscible organic solvent According to the present invention, the concentration of organic solvent at the point of mixing, which also is the final concentration prior to solvent removal (e.g. lyophilization), is 5%-30%, more preferred 10%-25%, most preferred 10%-25%. Typically, the lower the concentration of solvent, the smaller the resultant lipid vesicle liposome particles. Hence, it was found that under the inventive apparatus and process that a concentration of 10% t-butanol resulted in a preparation of liposomes where about 99% of the liposomes were less than 100 nm in size, compared to 20% t-butanol which created a preparation where 99% of the liposomes were less than 200 nm in size. A t-butanol concentration of 24% for example produced liposomes that were less than 400 nm in size. Accordingly, the mean particle size of the population of liposomes can be modulated by adjusting the concentration of solvent in the solvent mix and by keeping this concentration constant.

It is desirable to produce liposomes that are smaller than about 200 nm in size because these can be readily sterile-filtered using clinically-approved 0.22 μm pore-sized filters. Thus, in one aspect of the present invention a preferred solvent concentration, particularly for t-butanol, is one that is not more than about 20%, in order to produce liposomes less than 200 nm that can be used with such filters.

Quickly dispersing the lipid/solvent mix in water can help to maintain a steady solvent concentration, thus maintaining the concentration of solvent to say about 20%.

### (2) Lipids

Preferred phospholipids capable of forming liposomes include, but are not limited to dipalmitoylphosphatidylcholine (DPPC), phosphatidylcholine (PC; lecithin), phosphatidic acid (PA), phosphatidylglycerol (PG), phosphatidylethanolamine (PE), phosphatidylserine (PS). Other suitable phospholipids further include distearoylphosphatidylcholine (DSPC), dimyristoylphosphatidylcholine (DMPC), dipalmitoylphosphatidyglycerol (DPPG), distearoylphosphatidyglycerol (DSPG), dimyristoylphosphatidylglycerol (DMPG), dipalmitoylphosphatidic acid (DPPA); dimyristoylphosphatidic acid (DMPA), distearoylphosphatidic acid (DSPA), dipalmitoylphosphatidylserine (DPPS), dimyristoylphosphatidylserine (DMPS), distearoylphosphatidylserine (DSPS), dipalmitoylphosphatidyethanolamine (DPPE), dimyristoylphosphatidylethanolamine (DMPE), distearoylphosphatidylethanolamine (DSPE). The most preferred lipid is DPPC.

It may be desirable to include a sterol in the lipid solution to help facilitate or modulate liposome formation. One particularly useful sterol in this regard is cholesterol. Cholesterol is not necessary to facilitate liposome formation, but it does modulate liposome properties (e.g stability.

### (3) Ratio of Flow Rates Between Lipid Solution and Water

Providing start and stop of water and lipid solution flow are simultaneous, ratio of water to lipid solution flow rate determines solvent concentration and, consequently, liposome size. The higher the solvent concentration is, the larger the formed liposomes will be. The ratio of water flow rate to lipid solution flow rates is preferably at least 2:1 (yielding an organic solvent concentration of not more than about 33⅓%), more preferably at least 3:1 (yielding an organic solvent concentration of not more than about 25%). It is preferably not more than 19:1. It may be between about 19:1 (achieving an organic solvent concentration of about 5%) and 3⅓:1 (achieving an organic solvent concentration of about 30%), more preferably between 9:1 (achieving an organic solvent concentration of about 10%), and 5:1 (achieving an organic solvent concentration of about 20%), or between 9:1 and 4:1 (achieving an organic solvent concentration of about 25%).

Accordingly, the flow rate of water according to the present invention may be about 1.7 liters per minute. The flow rate of lipid/solvent according to the present invention may be about 0.43 liters per minute. Flow rate can be adjusted as practical for a given desired liposome size, as long as ratio is kept constant. Thus, for example, if it is desired to produce a liposome preparation where more than about 99% of liposomes are of a size less than about 200 nm, and the concentration of organic solution concentration is about 20%, then flow rates can be adjusted, while keeping a ratio of water flow rate to lipid solution flow rate of about 4-to-1, according to practical considerations such as practical mixing time and volume of solutions to be used.

### (4) Temperature of the Liquids

The preferred minimum temperature is related to the transition temperature. It is desirable to heat both the water and lipid solution liquids of the present invention; preferably to 10° C. or more above the transition temperature for components. Thus, it may be 10, 11, 12, 13, 14, 15, 16, 17, 18, 19, 20 or more degrees above the transition temperature. The liquids can be heated whilst in their respective holding tanks, which can be insulated with jackets to reduce heat loss. The temperature of either liquid may be about 40° C.-45° C., about 45° C.-50° C., about 50° C.-55° C., or about 55° C.-60° C. For DPPC the temperature is preferably at least 42° C., more preferably at least 45° C., most preferably at least 50° C. The maximum temperature is not critical, but of course higher temperatures necessitate greater energy inputs. For DPPC, the temperature chosen is preferably between about 42° C. and 65° C., more preferred 45° C. to 60° C., most preferred 50° C. to 55° C.

### (5) Cooling

Many processes require bulk to be cooled prior to storage, filtration or other processing. It is our surprising observation that, at the temperature and solvent concentration required for the liposome forming step of our process, liposome size increases with time following formation of liposomes. Consequently, if cooling occurs in the collecting vessel, batch size affects final size of liposomes, as larger batches take longer to cool. Instant cooling, made feasible by the use of a heat exchanger immediately following formation of liposomes, allows for control of liposome size and removes this obstacle to batch size independence. In order to maintain liposome size cooling time should not exceed 20° C. in 5 hours, e.g. cooling from about 55° C. to about 35° C. in less than 5 hours, more preferred from about 55° C. to about 30° C. in less than 2 hours, most preferred from about 55° C. to about 30° C. in less than 30 minutes. The mixture may be cooled to lower temperatures if desired.

(6) Continuous Flow of Each Liquid into Each Other

The liquids of the present invention, i.e., water and lipid solution, can be pumped under separate motors that are set or adjusted according to desirable flow rates as described above, and stored in large vats that can hold many liters of each liquid. Thus, a tank that holds up to 50 L or more (preferred 200 L) of water-for-injection can be used as a reservoir from which water can be pumped through the denoted pipes and T-junction arrangement, the rate of which can be monitored by placing a flow meter in the path of the water flow. Likewise, a separate tank holding many liters of the lipid/solvent solution, e.g., up to 50 L or more, can be pumped through the apparatus and also monitored for flow rate the same way.

Depending on the rate at which water is pumped through the apparatus, more or less water will be depleted from the holding tank over a certain period of time. The same obviously applies to the lipid solution reservoir. Since the rate of water flow is sometimes desired to be at least about four times that of the flow rate for lipid solution, it would be desirable to use a holding tank that can accommodate at least four times the volume of water than the lipid solution volume. Certainly, however, there will be a period of time where there is sufficient liquid in both holding tanks to produce a continuous flow of water and lipid solution during that period of time to maximize the quantity of appropriately-sized liposomes that can be produced per unit time. A “tank” may be any vessel capable of holding and/or heating the volumes of liquids discussed herein, including, but not limited to, vessels made from glass, stainless steel and plastic.

### (7) Unobstructed Flow of Liquids, and Turbulence-Inducing Means

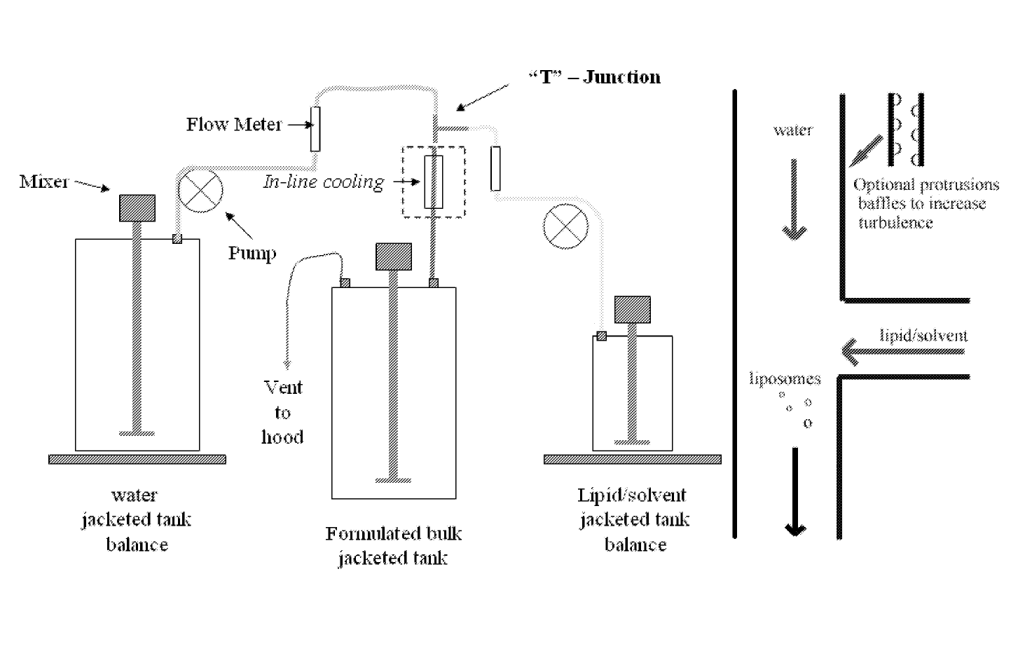

As mentioned above, a useful arrangement for introducing lipid solution into a stream of water is via two pipes oriented in such a way that the interiors of each pipe are open to one another at the site where they abut, i.e., at the junction, without any internal obstruction between the two openings that would otherwise prevent the bulk of the lipid solution from flowing freely through that opening. The two streams can meet at any angle, thus the pipes through which water and lipid solution, respectively, flow might meet at about 90 degrees, or less than 90 degrees See FIG. 1.

Because the present method is highly adaptable and readily scalable for commercial manufacturing purposes, any diameter of pipes may be used depending on appropriate modification of other parameters, such as flow rates and solvent concentration, according to the present invention. Accordingly a pipe of the present invention may be of any diameter, such as of a diameter about 1 mm, 2 mm, 3 mm, 4 mm, 5 mm, 6 mm, 7 mm, 8 mm, 9 mm, 10 mm, 12 mm, 13 mm, 14 mm, 15 mm, 16 mm, 17 mm, 18 mm, 19 mm, or 20 mm, or greater than a 20 mm diameter. The diameter may be chosen after consideration of the flow rate and mixing efficiency.

A pipe of such diameter may be uniform across its entire length or over part of its length. That is, in order to accommodate typical “tubing” connectors that are widely used in laboratories to facilitate joining of glass pipings to one another or to taps or pumps in a flexible manner, a pipe of the present invention may narrow at one terminal end to ease the insertion into such a tube.

The two pipes that make up the junction may or may not be of the same diameter at the junction where their openings meet. Thus, the water-bearing pipe may be narrower or wider than the lipid solution pipe, or vice versa. A pipe of the present invention may be glass, plastic, or metal.

It is possible to use pipes whose internal surfaces contain ridges, baffles, indentations, or protrusions that modulate the flow of liquid through their internal hollow core. If it is desirous to increase the turbulence of the environment at the site where water meets lipid solution, then one of these such pipes can be used to excite the flow of water to create a turbulent flow at the junction and thereby induce higher than normal shear forces to facilitate liposome formation. The protrusions or baffles could optimally be placed “upstream” of the junction in the water-flowing pipe, as well as, or instead of below the junction, to facilitate the mixing of the liquids.

### (8) Other Considerations, Ingredients, and Parameters

(i) Liposomes

It is desirable to produce liposomes that are smaller than 200 nm in size because these can be readily sterile-filtered using clinically-approved 0.22 μm pore-sized filters. A preparation that is made according to the present method using the inventive apparatus comprises a population of liposomes of a particular maximum size

In general, there is an increase in liposome size with decreased ratio of water flow rate to lipid solution flow rate and thus with increased organic solvent concentration. Liposome size may also be affected by other factors such as temperature or organic solvent used.

The liposomes that are produced after the lipid/solvent converges and mixes with the water then can optionally pass through a cooling jacket and be collected in a separate tank. That preparation of liposomes may then be lyophilized and later reconstituted according to well-known methods.

(ii) Bioactive Agents

MUC-1 is a large mucin that contains a polypeptide core consisting of 30-100 repeats of a 20 amino acid sequence. MUC-1 peptides, glycopeptides, lipopeptides and glycolipopeptides are particularly desirable peptides for incorporation into liposomes of the present invention, but the present invention is not limited to only these substances, since any other peptide, bioactive agent, drug, or therapeutic compound can be incorporated into a liposome of the present invention.

Preferably, the agent is a peptide (optionally glycosylated and/or lipidated) which comprises at least five, at least six, at least seven, at least eight, or at least nine, consecutive residues of the aforementioned 20 amino acid repeat sequence. It should be appreciated that since this is a tandem repeat, the choice of which amino acid is the first one is essentially arbitrary. Preferably, the peptide comprises at least the DTR tripeptide of the repeat sequence. It may comprise e.g., the PDTRP (AAs 13-17 of SEQ ID NO:1), SAPTDRP (AAs 12-17), TSAPDTRP (AAs 11-17), PDTRPAP (AAs 13-19) or TSAPDTRPAP (AAs 11-19) sequences. The agent may comprise more than one repeat, and it may comprise a non-integer number of repeats, e.g., 1¼.

Lipidation facilitates incorporation of the peptide into liposome. Preferably, if lipidated, the peptide comprises or consists of a first sequence which is a fragment of the tandem repeat region (which fragment may be less than, equal to, or more than a single repeat) and a second sequence that is lipidated. The first sequence is preferably the MUC1-derived sequence of BLP25 or BLP40 as described below.

The second sequence is preferably attached to the C-terminal of the first sequence, and is preferably not more than five amino acids, and most preferably is two or three amino acids. Preferably one to three of the amino acids are lipidated, and preferably these are consecutive. Preferably, the lipidated amino acids are, independently, Ser*, Thr, Asp, Glu, Cys, Tyr, Lys*, Arg, Asn, or Gln (*best). Preferably, the final amino acid of the second sequence is not lipidated, and preferably it is Gly*, Ala, Val, Leu*, or Ile. Preferably the lipid group is a C12 (lauric), C14 (myristic), C16 (palmitic)*, C18 (stearic) or C20 (arachidic) lipid.

With respect to MUC-1, an agent of particular interest is the 27 amino acid lipopeptide, “BLP25”. This consists of a 25-amino acid residue portion of the trnadem repeat region of the MUC-1 protein (i.e., 1¼ repeats) and a two amino acid C-terminal extension (KG), in which the K (lysine) is lipidated as shown below:

(SEQ ID NO: 1)
STAPPAHGVTSAPDTRPAPGSTAPP-K(palmitoyl)-G-OH

Another agent of particular interest, “BGLP40”, comprises a 40 aa residue fragment of the tandem repeat region of the MUC-1 protein, and a C-terminal extension (SSL) and which is lipidated as shown below (glycosylation shown is an example and other glycosylation patterns as well as no glycosylation is included):

(SEQ ID NO: 2)
TSAPDTRPAPGS(Tn)T(Tn)APPAHGVTSAPDT(Tn)RPAPGSTAPPA
HGVS(Lipo)S(Lipo)L

(iii) Other Ingredients

Further suitable ingredients of the lipid component are glycolipids and other lipid adjuvants, such as monophosphoryl lipid A (MPLA) or Lipid A, or synthetic adjuvants that may or may not be analogs of naturally occurring adjuvants.

(iv) Water

Clinical grade water.

### (9) Scalability

Volumes are only limited by vessel size. Commercial processes could be computer controlled.

1-25. (canceled)

26. A composition comprising liposomes of constrained particle size wherein at least about 90% of the liposomes are of a particle size less than about 200 nm and wherein the liposomes further comprise a MUC-1 peptide, or a glycosylated and/or lipidated derivative of a MUC-1 peptide.

27. A composition according to claim 26, wherein the liposomes comprise phospholipids selected from the group consisting of dipalmitoylphosphatidylcholine (DPPC), phosphatidylcholine (PC; lecithin), phosphatidic acid (PA), phosphatidylglycerol (PG), phosphatidylethanolamine (PE), phosphatidylserine (PS). Other suitable phospholipids further include distearoylphosphatidylcholine (DSPC), dimyristoylphosphatidylcholine (DMPC), dipalmitoylphosphatidyglycerol (DPPG), distearoylphosphatidyglycerol (DSPG), dimyristoylphosphatidylglycerol (DMPG), dipalmitoylphosphatidic acid (DPPA); dimyristoylphosphatidic acid (DMPA), distearoylphosphatidic acid (DSPA), dipalmitoylphosphatidylserine (DPPS), dimyristoylphosphatidylserine (DMPS), distearoylphosphatidylserine (DSPS), dipalmitoylphosphatidyethanolamine (DPPE), dimyristoylphosphatidylethanolamine (DMPE) and distearoylphosphatidylethanolamine (DSPE).

28. A composition according to claim 26, wherein the liposomes comprise dipalmitoylphosphatidylcholine (DPPC).

29. A composition according to claim 26, wherein the liposomes further comprise a sterol.

30. A composition according to claim 26, wherein the MUC-1 peptide comprises an amino acid sequence of SEQ ID NO:1.

31. A composition according to claim 30, wherein the MUC-1 peptide is lipidated at a lysine.

32. A composition according to claim 30, wherein the MUC-1 peptide is palmitoylated.

33. A composition according to claim 26, wherein the MUC-1 peptide comprises an amino acid sequence of SEQ ID NO:2.

34. A composition according to claim 33, wherein the MUC-1 peptide is lipidated at the two final serines of SEQ ID NO:2.

35. A composition according to claim 33, wherein the MUC-1 peptide is glycosylated.

36. A composition according to claim 26 wherein the composition is lyophilized.

37. A composition that is reconstituted from the lyophilized composition of claim 36.

38. A composition according to claim 26, wherein the composition is obtained by a process comprising the steps of:
providing a substantially continuously flowing stream of water,
providing a substantially continuously flowing stream of an organic solvent, said organic solvent containing, dissolved therein, at least one lipid and at least one MUC-1 peptide, the lipid or lipids being capable of forming liposomes, substantially continuously mixing said stream of water and the stream of organic solvent, so as to obtain a mixture, cooling the mixture, and
allowing liposomes to form within the mixture,
wherein the ratio of the flow rate of the stream of water to the flow rate of the stream of organic solvent, and the rate of cooling of said mixture, are controlled so as to obtain a preparation of liposomes such that at least about 90% of the liposomes are of a particle size less than about 200 nm.

39. A composition according to claim 38, wherein the ratio of the flow rate of the stream of water to the flow rate of the stream of organic solvent is at least about 2:1.

40. A composition according to claim 38, wherein the rate of cooling is on average at least about 4° C. per hour.

41. A composition according to claim 38, wherein the organic solvent stream is, prior to the mixing, at a temperature at least 10° C. above the transition temperature of said lipids.

42. A composition according to claim 38, further comprising providing means for inducing turbulence in the stream of water, the stream of organic solvent, or in a stream of mixture resulting from the mixing.

43. A vaccine formulation comprising a sterile filtered composition of claim 26.

44. A vaccine formulation comprising a lyophilized composition of claim 43.

45. A vaccine formulation comprising a reconstituted composition of claim 44.

46. A method for producing a preparation of liposomes of constrained particle size, said method comprising the steps of
providing a substantially continuously flowing stream of water,
providing a substantially continuously flowing stream of an organic solvent, said organic solvent containing, dissolved therein, at least one lipid, said lipid or lipids being capable of forming liposomes, substantially continuously mixing said stream of water and said stream of organic solvent, so as to obtain a mixture,
cooling said mixture, and
allowing liposomes to form within said mixture,
the ratio of the flow rate of the stream of water to the flow rate of the stream of organic solvent, and the rate of cooling of said mixture, being controlled so as to obtain a preparation of liposomes such that at least about 90% of the liposomes are of a particle size less than about 200 nm.
